(19) United States
(12) Patent Application Publication
(10) Pub. No.: US 2023/0363811 A1
(43) Pub. Date: Nov. 16, 2023

(54) GALLBLADDER CRYOABLATION DEVICE AND METHOD

(71) Applicant: Arizona Board of Regents on Behalf of the University of Arizona, Tucson, AZ (US)

(72) Inventor: Charles T. HENNEMEYER, Marana, AZ (US)

(21) Appl. No.: 18/028,305

(22) PCT Filed: Sep. 23, 2021
(86) PCT No.: PCT/US2021/051708

§ 371 Date: Mar. 24, 2023

Related U.S. Application Data
(60) Provisional application No. 63/083,213, filed on Sep. 25, 2020.

(51) Int. Cl. A61B 18/02

(52) U.S. Cl. CPC A61B 18/02; A61B 2018/0212; A61B 2018/00535

(57) ABSTRACT

A gallbladder cryoablation device can include an elongated body and a compliant thermal conductive wire. The elongated body includes a sharp distal end for insertion into a gallbladder, a liquid duct configured to insert a cryoablation fluid into the gallbladder, and an air duct configured to evacuate any gas displaced by the cryoablation fluid. The compliant thermal conductive wire is configured to expand from the elongated body and contact an inner wall of the gallbladder. A method of using the gallbladder cryoablation device includes inserting the elongated body, via the sharp distal end of the elongated body, into the gallbladder; positioning the compliant thermal conductive wire into the gallbladder until a sufficient portion of the compliant thermal conductive wire contacts the inner wall of the gallbladder; directing the cryoablation fluid into the cryoablation device; and cooling the compliant thermal conductive wire to a freezing temperature.

### BACKGROUND

Gallstones are hardened deposits of digestive fluid that can form in the gallbladder. Gallstones can cause blockage of the cystic duct, which connects the gallbladder to the duodenum of the small intestine. This blockage can cause cholecystitis, leading to the need of gallbladder removal surgery (e.g., a cholecystectomy). However, many patients are not candidates for a cholecystectomy due to underlying health conditions; and even laparoscopic cholecystectomy has potential complications and an extended recovery time. Gallbladder cryoablation is an alternative procedure for these patients, however, this procedure currently uses devices that were designed for other purposes and therefore requires highly skilled surgeons and a relatively long time to complete the procedure. Furthermore, existing gallbladder cryoablation can still miss portions of the gallbladder due to, for example, the gallstones themselves, rendering the gallbladder cryoablation to cure the cholecystitis a failure.

### BRIEF SUMMARY

A gallbladder cryoablation device and methods of use are provided. The embodiments described herein provide targeted cryoablation that freezes the gallbladder and the proximal cystic duct without damaging surrounding anatomical structures or tissues (e.g., creating a hole in the duodenum). Advantageously, the described cryoablation device and methods of use can provide a minimally invasive way of curing cholecystitis, which may otherwise be impossible for patients with underlying conditions.

A gallbladder cryoablation device can include an elongated body and a compliant thermal conductive wire associated with the elongated body. The elongated body includes a sharp distal end for insertion into a gallbladder, a liquid duct configured to direct a cryoablation fluid to a vaporization area of the elongated body, and an air duct configured to evacuate cryoablation gas formed at the vaporization area. The compliant thermal conductive wire is configured to extend from the elongated body and contact an inner wall of the gallbladder. In some cases, the compliant thermal conductive wire is disposed between a proximal non-compliant band of thermal conductive material and a distal non-compliant band of thermal conductive material.

A method of using the gallbladder cryoablation device can include inserting the elongated body, via the sharp distal end of the elongated body, into a gallbladder; positioning the compliant thermal conductive wire into the gallbladder until a sufficient portion of the compliant thermal conductive wire contacts the inner wall of the gallbladder; directing the cryoablation fluid into the cryoablation device; and cooling the compliant thermal conductive wire to a freezing temperature resulting in the gallbladder becoming frozen from the contact of the sufficient portion of the compliant thermal conductive wire to the inner wall of the gallbladder without damaging surrounding anatomical structures or tissues.

In some cases, the gallbladder cryoablation device is used with a medical balloon. The medical balloon can be positioned around the elongated body and in fluid communication with liquid duct of the elongated body. In some cases, the compliant thermal conductive wire is positioned around the medical balloon. The medical balloon can also be in fluid communication with the air duct of the elongated body and configured to evacuate any cryoablation gas formed in the medical balloon.

A method of using the gallbladder cryoablation device with the medical balloon includes inserting the elongated body, via the sharp distal end of the elongated body, into the gallbladder; filling the medical balloon with cryoablation fluid, via the liquid duct, until a sufficient portion of the compliant thermal conductive wire contacts the inner wall of the gallbladder; and cooling the compliant thermal conductive wire to a freezing temperature, resulting in the gallbladder becoming frozen from the contact of the sufficient portion of the compliant thermal conductive wire to the inner wall of the gallbladder without damaging surrounding anatomical structures or tissues.

In some cases, a compliant thermally conductive wire can be in the form of a coil that includes a compliant inner mandrel having a longitudinal length and a thermally conductive outer material coupled to the compliant inner mandrel along the longitudinal length of the compliant inner mandrel. In some cases, the compliant inner mandrel is nitinol or para-aramid. In some cases, the thermally conductive outer material is silver. In some cases, the compliant inner mandrel includes a plurality of pre-programmed loops configured to cause the thermally conductive outer material to contact a sufficient portion of an inner wall of a gallbladder to freeze the gallbladder. In some cases, the coil further includes a sharp distal end coupled to a distal end of the compliant inner mandrel or a distal end of the thermally conductive outer material, wherein the introducer is configured to pierce a gallbladder of a patient.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A illustrates a problem with certain existing gallbladder cryoablation devices and methods.

FIG. 1B illustrates a cross-sectional view of a gallbladder having gallstones and a deployed cryoablation device in accordance with an example embodiment.

FIGS. 2A-2C illustrate an embodiment of a cryoablation device with compliant thermal conductive wire disposed between two non-compliant bands of thermal conductive material.

FIGS. 3A-3D illustrate an embodiment of a cryoablation device with compliant thermal conductive wires positioned around a medical balloon.

FIGS. 4A-4F illustrate an embodiment of a cryoablation device configured to provide extension of compliant thermal conductive wire into a gallbladder.

FIG. 5 illustrates a side-angle view of a cryoablation device that has positioned compliant thermal conductive wire against an inner wall of a gallbladder.

FIGS. 6A-6D illustrate a cryoablation device that has varying diameters of compliant thermal conductive wire for positioning against different portions of an inner wall of a gallbladder.

FIGS. 7A-7F illustrate various implementations of a compliant thermal conductive wire in the form of a coil.

FIG. 8A illustrates a method of using a cryoablation device.

FIG. 8B illustrates a method of using a cryoablation device with a medical balloon.

### DETAILED DESCRIPTION

A gallbladder cryoablation device and methods of use are provided. The embodiments described herein provide targeted cryoablation that freezes the gallbladder without damaging surrounding anatomical structures or tissues (e.g., creating a hole in the duodenum). Advantageously, the described cryoablation device and methods of use can provide a minimally invasive way of curing cholecystitis, which may otherwise be impossible for patients with underlying conditions.

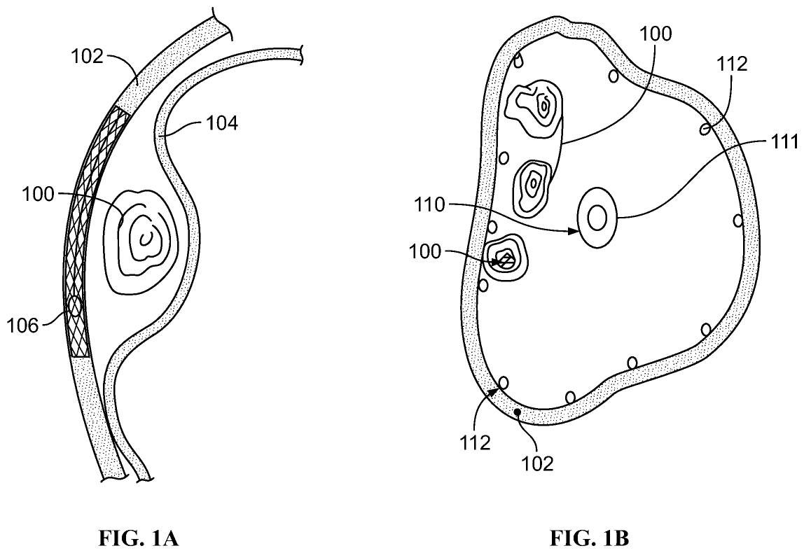

FIG. 1A illustrates a problem with certain existing gallbladder cryoablation devices and methods. Referring to the partial cross-section shown FIG. 1A, a gallstone 100 may be located against a gallbladder wall 102. It is desirable to perform cryoablation in a manner that directs the freezing along the gallbladder wall 102. When using a medical balloon 104, for example, filled with cryoablation fluid, the medical balloon 104 contacts the gallbladder wall 102, except in non-contact area 106 created by the gallstone 100. The non-contact area 106 is spared cryoablation/freezing caused by the medical balloon 104, resulting in failure of the procedure to treat/cure cholecystitis. In other cases, a cryoprobe may be used to form an ice ball from fluid within the gallbladder to freeze a portion(s) of the gallbladder wall 102, however, due to the difficultly in controlling the position, size, and shape of the ice ball formed by existing cryoprobes, the procedure often fails to treat/cure cholecystitis. In some cases, to account for the difficulty in controlling the position, size, and shape of the ice ball formed by existing cryoprobes, multiple cryoprobes are used to create multiple ice balls and/or a single large ice ball, which creates further risk of damage to the surrounding anatomical structure(s) and tissue(s).

FIG. 1B illustrates a cross-sectional view of a gallbladder having gallstones and a deployed cryoablation device in accordance with an example embodiment. Referring to FIG. 1B, three gallstones 100 are shown. A gallbladder cryoablation device 110 in accordance with an example embodiment can include an elongated body (cross-section shown at center and referenced with 111) and a compliant thermal conductive wire 112 (shown as a plurality of points in cross-section) associated with the elongated body 111. The compliant thermal conductive wire 112 is configured to extend from the elongated body 111 and contact an inner wall 102 of the gallbladder. When deployed in a gallbladder, the compliant thermal conductive wire 112 is positioned at various points of the gallbladder wall 102.

As can be seen in comparison to the existing device shown in FIG. 1A, a gallstone 100 does not interfere with the compliant thermal conductive wire 112 coming into contact with the inner wall 102 of the gallbladder. Indeed, the compliant thermal conductive wire 112 even fits between a plurality of gallstones 100. Due to the much higher thermal conductivity and the ability to position the compliant thermal conductive wire 112 relative to existing solutions (e.g., a medical balloon filled with cryoablation fluid described in FIG. 1A), cryoablations/freezing all and/or any specific portion the gallbladder wall 102 is possible (e.g., there is no non-contact area 106 that would be spared cryoablation/freezing). For instance, thermal conductivity (i.e., watts/(meter*kelvin)) of silver is approximately 430, copper is approximately 300, and diamond is approximately 1000. On the other hand, thermal conductivity of water is approximately 0.59 and ice is approximately 1.6. Therefore, cryoablation is much more efficient using a highly thermally conductive material (e.g., silver, copper, or diamond) as opposed to only using only existing systems and methods to freeze tissue.

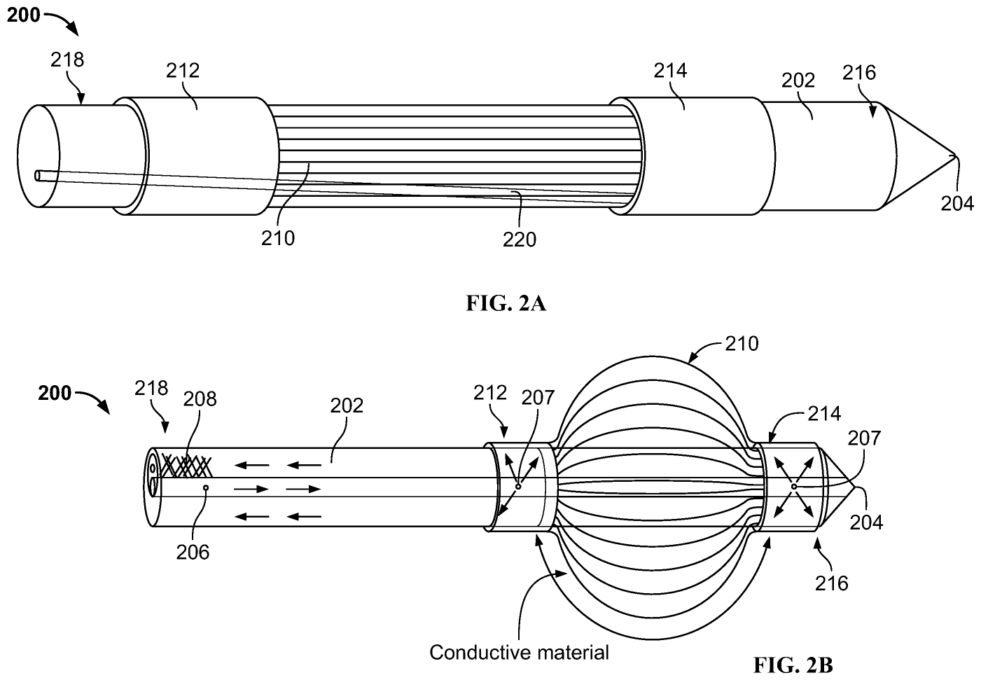

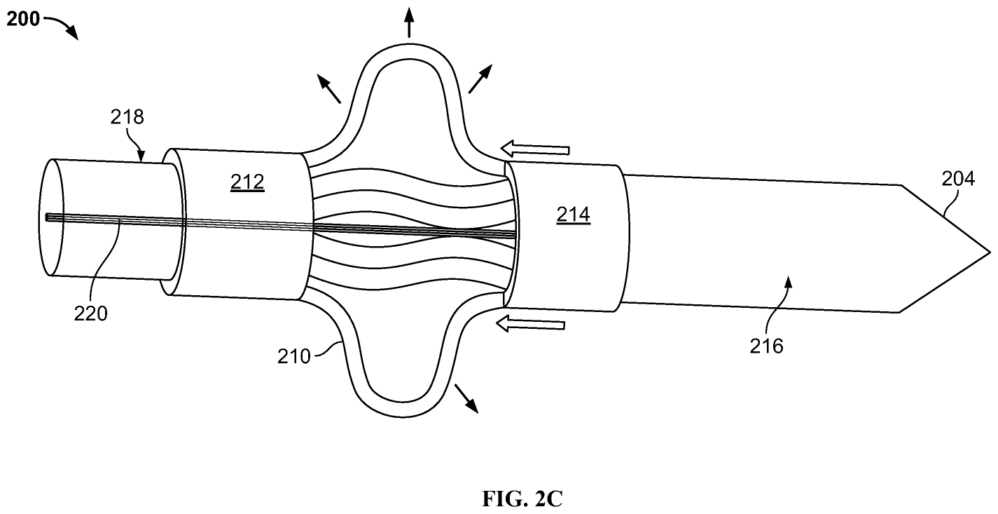

FIGS. 2A-2C illustrate an embodiment of a cryoablation device with compliant thermal conductive wire disposed between two non-compliant bands of thermal conductive material. Referring to FIGS. 2A-2C, a gallbladder cryoablation device 200 includes an elongated body 202 (e.g., a cryoprobe) and a compliant thermal conductive wire 210 associated with the elongated body 202 and configured to expand to have sufficient diameter to contact an inner wall of the gallbladder. As can be seen in FIG. 2B, the elongated body 202 has a sharp distal end 204 for insertion into a gallbladder, a liquid duct 206 configured to direct a cryoablation fluid to the vaporization areas 207, and an air duct 208 configured to evacuate the gas that results from the vaporization of the cryoablation fluid during cooling. The vaporization areas 207 are area(s) in which the cryoablation fluid turns to a gas, cooling the compliant thermal conductive wire 210 of the gallbladder cryoablation device 200. In some cases, the cryoablation fluid is argon. In some cases, the cryoablation fluid is another gas suitable for use as cryoablation fluid (e.g., inert gases). In the illustrated embodiment, the compliant thermal conductive wire 210 is disposed between a proximal non-compliant band 212 of thermal conductive material and a distal non-compliant band 214 of thermal conductive material. In the illustrated embodiment, the compliant thermal conductive wire 210 is arranged in equidistant sections, forming a cylindrical shape. However, in other implementations, the compliant thermal conductive wire 210 may be in spirals, weaves, and non-equidistant section configurations. In some cases, the ends of the wire 210 (e.g., each section) can be respectively coupled to the two bands of thermal conductive material 212, 214. In some cases, the wire 210 and both bands of thermal conductive material 212, 214 are formed of a same material and may be formed as a single body.

Referring to FIGS. 2A and 2C, the distal non-compliant band 214 of thermal conductive material can be slidably attached to the elongated body 202 and located at a distal portion 216 of the elongated body 202 while the proximal non-compliant band 212 of thermal conductive material is affixed to the elongated body 202 at a proximal portion 218 of the elongated body 202. In operation, the distal non-compliant band 214 of thermal conductive material is slidable from the distal portion 216 of the elongated body 202 towards the proximal portion 218 of the elongated body 202, which causes the compliant thermal conductive wire 210 to deform outwardly (e.g., towards the inner wall of a gallbladder). The distal non-compliant band 214 of thermal conductive material can be slid along the elongated body 202, for example, via a tether element 220 attached to the distal non-compliant band 214 of thermal conductive material and that is pulled back.

As shown in FIG. 2C, the distal non-compliant band 214 of thermal conductive material can be slid along the elongated body 202, causing the compliant thermal conductive wire 210 to deform until sufficient contact is made between the compliant thermal conductive wire 210 and the inner wall of the gallbladder to ablate/freeze the gallbladder. The tether element 220 may be attached to the distal non-compliant band 214 of thermal conductive material such that, when the tether element 220 is pulled towards the proximal portion 218 of the elongated body 202, the compliant thermal conductive wire 210 is deformed until sufficient contact is made between the compliant thermal conductive wire 210 and the inner wall of the gallbladder to ablate/freeze the gallbladder.

Referring to FIGS. 2A and 2B, the proximal non-compliant band 212 of thermal conductive material can be slidably attached to the elongated body 202 and located at the proximal portion 218 of the elongated body 202 while the distal non-compliant band 214 of thermal conductive material is affixed to the elongated body 202 at the distal portion 216 of the elongated body 202. In operation, the proximal non-compliant band 212 of thermal conductive material is slidable from the proximal portion 218 of the elongated body 202 towards the distal portion 216 of the elongated body 202, which causes the compliant thermal conductive wire 210 to deform outwardly (e.g., towards the inner wall of a gallbladder).

The proximal non-compliant band 212 of thermal conductive material can be slid along the elongated body 202, for example, via a rigid tether element (not shown) attached to the proximal non-compliant band 212 of thermal conductive material. The proximal non-compliant band 212 of thermal conductive material can be slid, via pushing the rigid tether element, along the elongated body 202, causing the compliant thermal conductive wire 210 to deform until sufficient contact is made between the compliant thermal conductive wire 210 and the inner wall of the gallbladder to ablate/freeze the gallbladder.

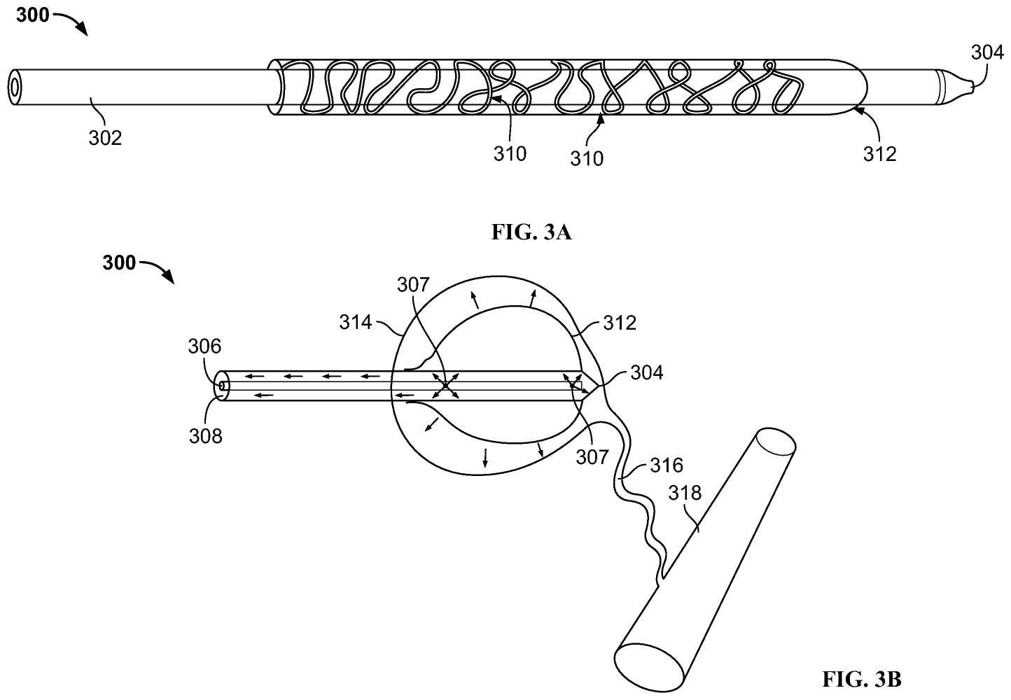

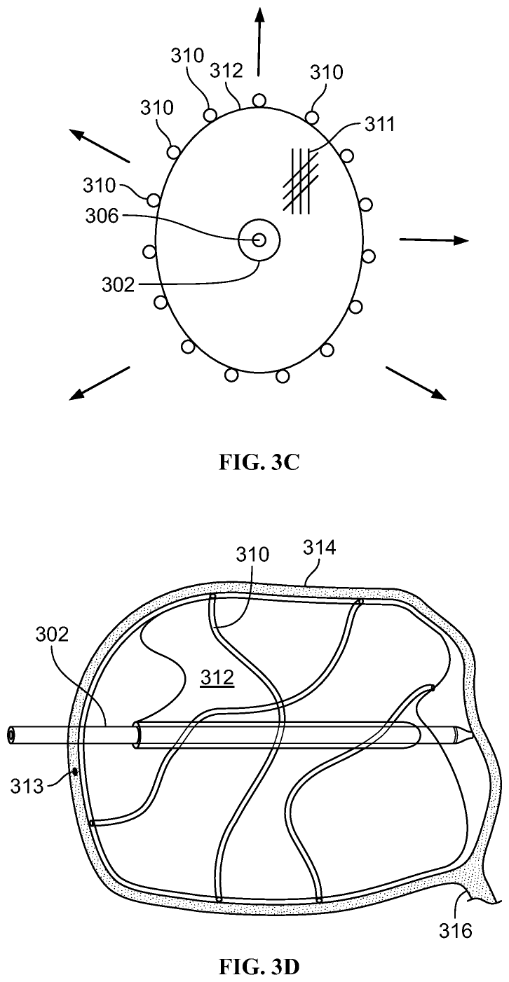

FIGS. 3A-3D illustrate an embodiment of a cryoablation device with compliant thermal conductive wires positioned around a medical balloon. Referring to FIGS. 3A-3D, a cryoablation device 300 includes an elongated body 302 (e.g., a cryoprobe) having a sharp distal end 304 for insertion into a gallbladder 314, a liquid duct 306 configured to direct a cryoablation fluid to a vaporization area 307, and an air duct 308 configured to evacuate cryoablation gas formed at the vaporization area 307. The cryoablation device 300 further includes a medical balloon 312 positioned around the elongated body 302 and in fluid communication with liquid duct 306 of the elongated body 302 and a compliant thermal conductive wire 310 positioned around the medical balloon 312. In some cases, the compliant thermal conductive wire 310 is configured to extend proportionally to match inflation of the medical balloon 312 when cryoablation fluid is inserted, via the liquid duct 306 of the elongated body 302, into the medical balloon 312. For example, the compliant thermal conductive wire 310 may include additional longitudinal length that extends and deforms to match a proportional position on the medical balloon 312 during the expansion of the medical balloon 312 while inflating.

The compliant thermal conductive wire 310 may be positioned around the medical balloon 312 in any way feasible to extend proportionally to match inflation of the medical balloon 312. For instance, the compliant thermal conductive wire 310 may be spaced evenly around the medical balloon 312, in a specific pattern (e.g., a cross-hatch weave) around the medical balloon 312, or in a random pattern around the medical balloon 312.

The medical balloon 312 can be filled with cryoablation fluid (e.g., in pressurized area 311 of medical balloon 312 until the compliant thermal conductive wire 310 has a sufficient portion in contact with the inner wall 313 of the gallbladder 314 to freeze the gallbladder 314 without damaging surrounding anatomical structures or tissues (e.g., freezing the proximal cystic duct 316 or the common bile duct 318). It should be understood that the cryoablation fluid may be in a liquid state or a gas state while inside the medical balloon 312. Furthermore, a surgeon may add cryoablation fluid via the liquid duct 306 (e.g., to expand the medical balloon 312 or further cool the compliant thermal conductive wire 310) or remove cryoablation fluid via the air duct 308 (e.g., to remove the cryoablation device 300) as needed.

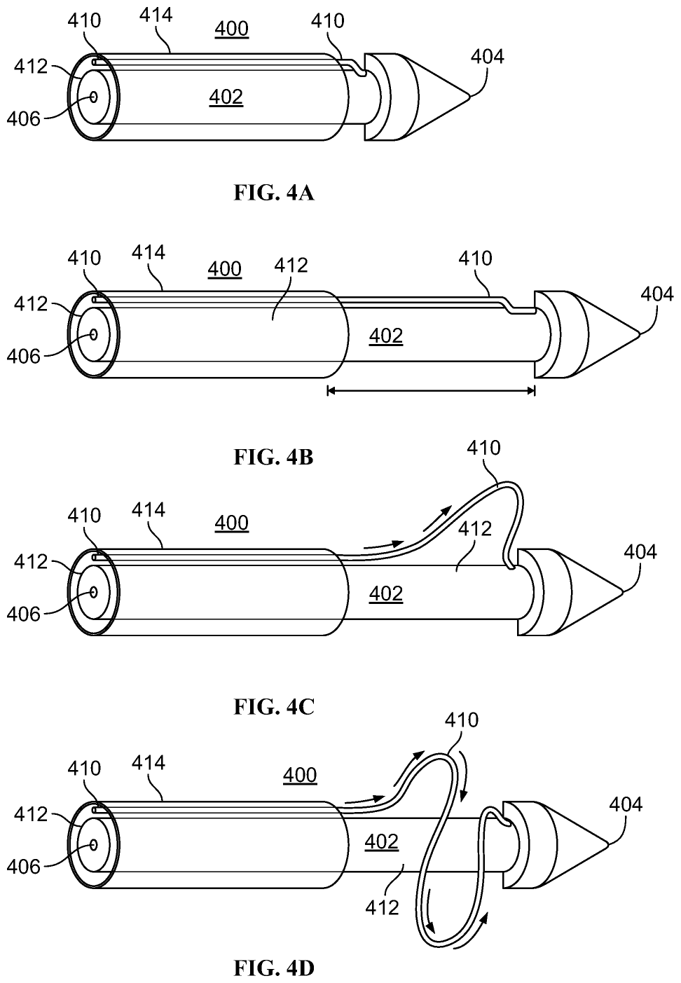

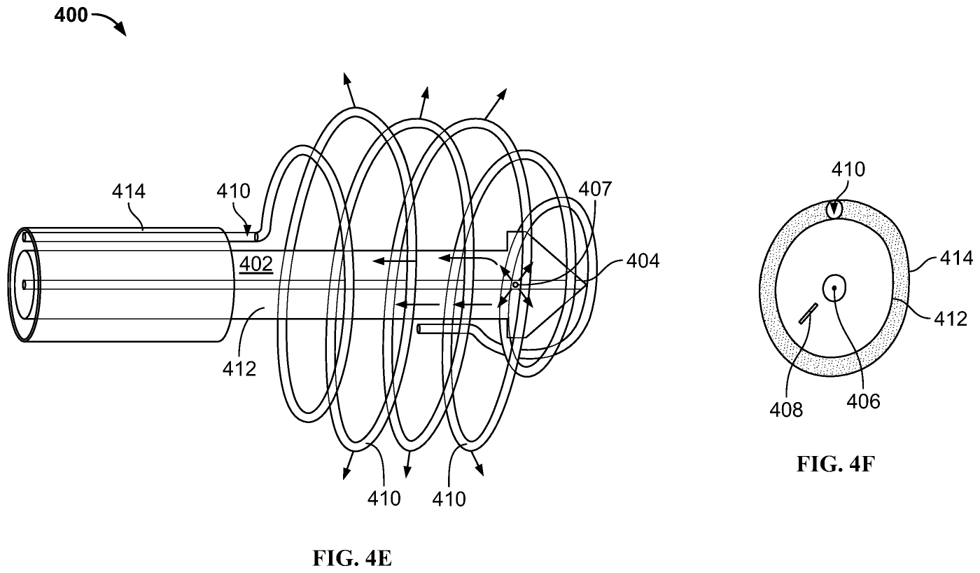

FIGS. 4A-4F illustrate an embodiment of a cryoablation device configured to provide extension of compliant thermal conductive wire into a gallbladder. Referring to FIGS. 4A-4F, a cryoablation device 400 includes an elongated body 402 (e.g., a cryoprobe) having a sharp distal end 404 for insertion into a gallbladder, a liquid duct 406 configured to direct a cryoablation fluid to a vaporization area 407 of the elongated body 402, and an air duct 408 configured to evacuate cryoablation gas formed at the vaporization area 407. In this embodiment, the elongated body 402 further includes an inner portion 412 and an outer portion 414. The inner portion 412 is extendable with respect to the outer portion 414. The compliant thermal conductive wire 410 is initially positioned longitudinally along a length of the of the elongated body 402 between the inner portion 412 and the outer portion 414.

Specifically, FIG. 4A illustrates a cryoablation device 400 in an initial or closed position. FIG. 4B illustrates the extension of the inner portion 412 with respect to the outer portion 414, exposing the compliant thermal conductive wire 410. This extendable feature is advantageous, for example, because it allows a surgeon to first insert the cryoablation device 400 into the gallbladder, via the sharp distal end 404; and then extend the inner portion 412 until another side of the gallbladder is reached by the sharp distal end 404. As illustrated in FIGS. 4B-4E, the compliant thermal conductive wire 410 can then be extended or pushed out from between the inner and outer portion 412, 414 of the elongated body 402 until a sufficient portion of the wire 210 contacts the inner wall of the gallbladder to ablate/freeze the gallbladder. Deployment of the cryoablation device 400 by a surgeon into the gallbladder, including the extension/pushing/deformation of the compliant thermal conductive wire 410 until a sufficient portion of the wire 410 contacts the inner wall of the gallbladder, may be aided by an imaging device (e.g., a computed tomography scanner).

FIG. 4F illustrates a cross-sectional view of the cryoablation device 400 including the outer portion 414, the inner portion 412, the compliant thermal conductive wire 410 positioned between the inner portion 412 and the outer portion 414, the liquid duct 406 configured to direct a cryoablation fluid to the vaporization area 407 of the elongated body 402, and the air duct 408 configured to evacuate cryoablation gas formed at the vaporization area 407. Although the liquid duct 406 and the air duct 408 are illustrated within the inner portion 412 in this embodiment, in some cases, the liquid duct 406 and/or the air duct 408 may be positioned outside the inner portion 412 (e.g., positioned between the inner portion 412 and the outer portion 414 or positioned and attached to the outer portion 414).

FIG. 5 illustrates a side-angle view of a cryoablation device that has positioned compliant thermal conductive wire against an inner wall of a gallbladder. A cryoablation device 500 includes an elongated body 502, a sharp distal end 504, and compliant thermal conductive wire 510. In some cases, the elongated body 502 is a catheter. In some cases, the elongated body 502 is coupled to the sharp distal end 504. A method of freezing a gallbladder 514 may include, for example, inserting the elongated body 502, via the sharp distal end 504, into the gallbladder 514, positioning the compliant thermal conductive wire 510 into the gallbladder until a sufficient portion of the compliant thermal conductive wire 510 contacts an inner wall of the gallbladder 514, directing cryoablation fluid into the cryoablation device 500, and cooling the compliant thermal conductive wire 510 to a freezing temperature, resulting in the gallbladder 514 becoming frozen from the contact of the sufficient portion of the compliant thermal conductive wire 510 to the inner wall of the gallbladder 514 without damaging surrounding anatomical structures or tissues. As illustrated in FIG. 5, the compliant thermal conductive wire 510 is contacting the inner wall 512 of the gallbladder 514. For example, with the aid of an imaging device, a surgeon can extend the compliant thermal conductive wire 510 until a sufficient portion contacts the inner wall 512 of the gallbladder 514 to freeze the gallbladder 514 without damaging surrounding anatomical structures or tissues (e.g., freezing the proximal cystic duct 516).

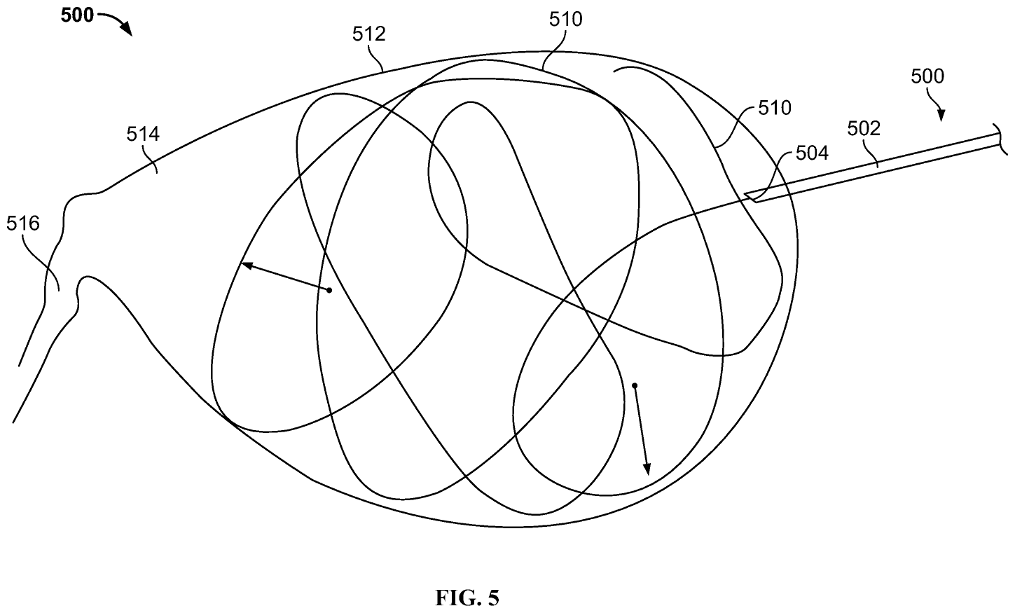

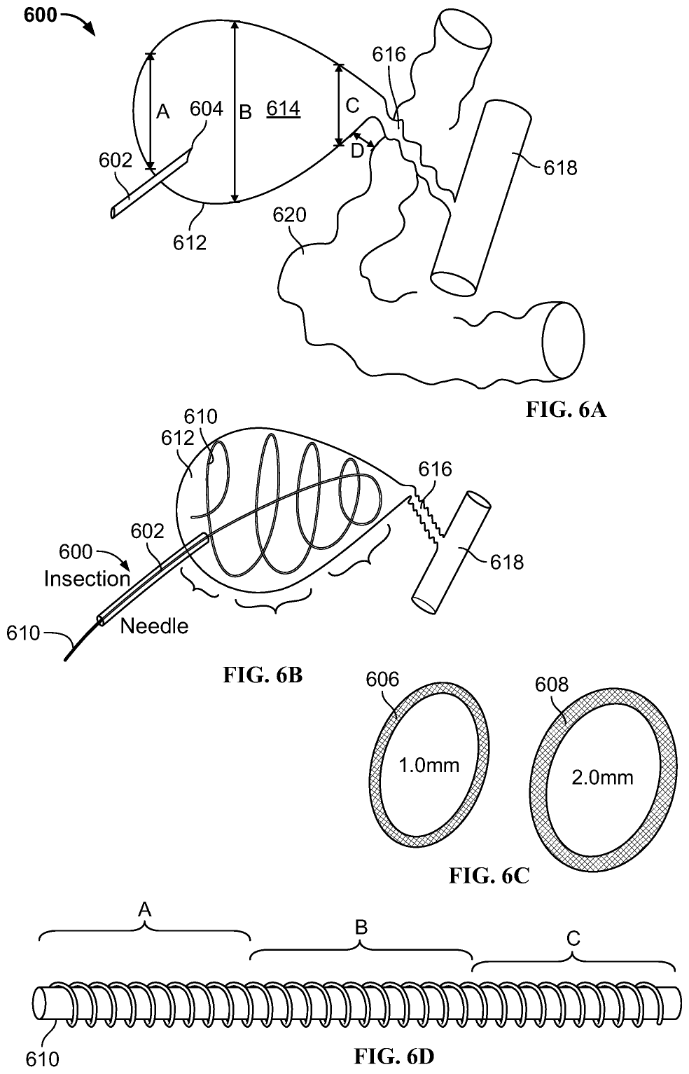

FIGS. 6A-6D illustrate a cryoablation device that has varying diameters of compliant thermal conductive wire for positioning against different portions of an inner wall of a gallbladder. Referring to FIGS. 6A-6D, a cryoablation device 600 includes an elongated body 602 having a sharp distal end 604 for insertion into a gallbladder 614. As illustrated in FIG. 6A, the gallbladder 614 has varying diameters across its inner wall 612 (e.g., A, B, and C). For example, a proximal inner wall A of the gallbladder 614 can have a diameter of approximately 1.5 centimeters, a mid-body inner wall B of the gallbladder 614 can have a diameter of approximately 2.5 centimeters, and a distal inner wall C of the gallbladder 614 can have a diameter of approximately 1.5 centimeters. It should be understood that the diameter of the inner wall 612 of the gallbladder 614 can be predetermined using medical imaging technology, including but not limited to, ultrasound imaging, magnetic resonance imaging, sonogram imaging, X-ray imaging, computed tomography imaging, and/or nuclear medicine imaging. Furthermore, a distance D between the end of the gallbladder 614/beginning of the cystic duct 616 and the duodenum 620 can also be predetermined using medical imaging technology.

Once the diameter of each portion A, B, C of the inner wall 612 of the gallbladder 614 is determined, as shown in FIG. 6D, the wire diameters for sections of the compliant thermal conductive wire 610 corresponding to each portion A, B, C of the gallbladder 614. These sections may be extruded and formed as a single wire of varying thickness. For example, a smaller diameter (e.g., portions A and C) of the inner wall 612 of the gallbladder 614 may require a 1.0 millimeter 606 compliant thermal conductive wire 610 for creating smaller loops (e.g., 1.5 centimeters) of contact around the inner wall 612 of the gallbladder 614 while a larger diameter (e.g., portion B) of the inner wall 612 of the gallbladder 614 may require a 2.0 millimeter 608 compliant thermal conductive wire 610 for creating larger loops (e.g., 2.5 centimeters) of contact around the inner wall 612 of the gallbladder 614. In other words, a smaller diameter compliant thermal conductive wire 610 (e.g., 1.0 millimeter diameter wire 606) may be more suited to provide a smaller loop, while a larger diameter compliant thermal conductive wire 610 (e.g., 2.0 millimeter diameter wire 608) may be more suited to provide a larger loop. In some cases, a compliant thermal conductive wire 610 may include a diameter of up to 4.0 millimeters.

Furthermore, the distance D can also influence the diameter of the compliant thermal conductive wire 610 that is positioned around the portion C of the inner wall 612 of the gallbladder 614 that is proximal to the cystic duct 616, the common bile duct 618, and the duodenum 620. For example, a relatively small distance D may require an even smaller diameter for the section of the compliant thermal conductive wire 610 corresponding to portion C as shown in FIG. 6D without damaging (e.g., freezing or creating a hole) in the proximal cystic duct 616, the common bile duct 618 and/or duodenum 620 while a relatively long distance D may allow for a larger diameter for the section of the compliant thermal conductive wire 610 corresponding to portion C as shown in FIG. 6D to ablate/freeze the gallbladder 614 without damaging (e.g., freezing or creating a hole) the proximal cystic duct 616, the common bile duct 618, and/or duodenum 620.

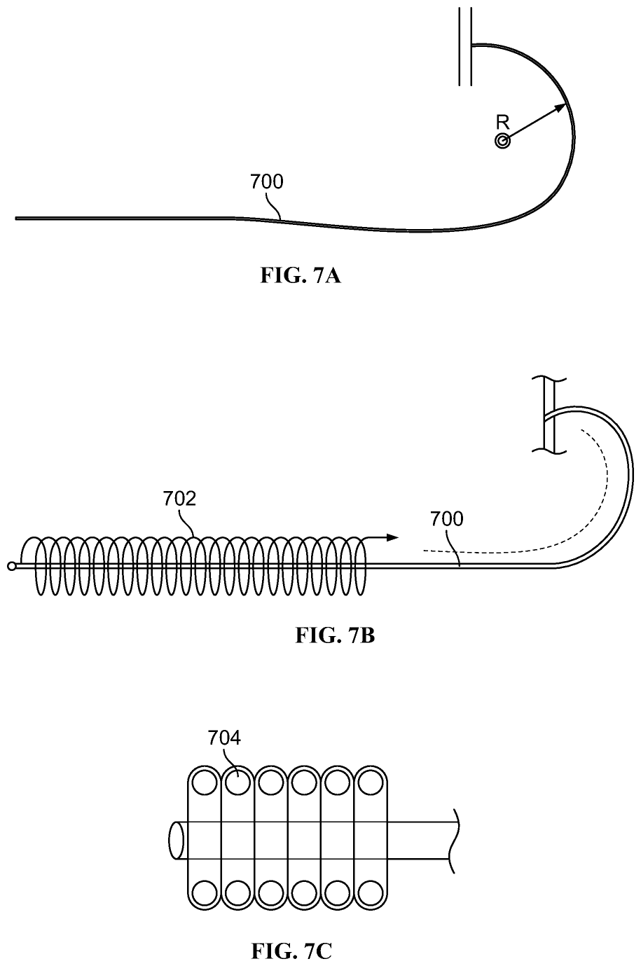

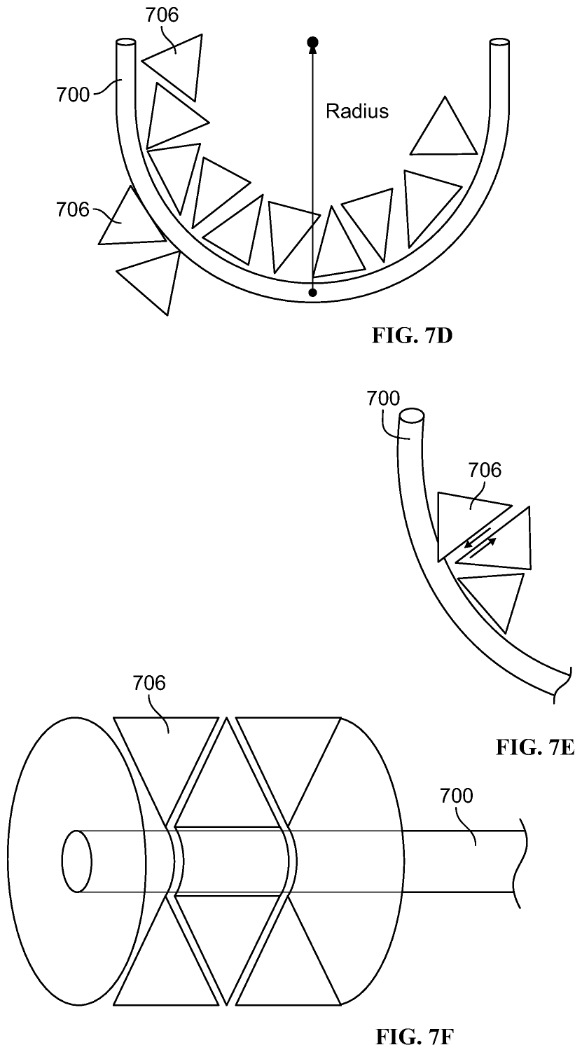

FIGS. 7A-7F illustrate various implementations of a compliant thermal conductive wire in the form of a coil. FIG. 7A shows a nitinol or para-aramid inner mandrel 700 that can be used, for example, as a guide to position a coil formed of a thermal conductive wire to contact a sufficient portion of the inner wall of a gallbladder to freeze the gallbladder without damaging surrounding anatomical structures or tissues due to its super-elastic and shape memory properties.

In some cases, the nitinol or para-aramid inner mandrel 700 may have one or more loops, each with a radius R pre-programmed into the nitinol or para-aramid inner mandrel 700 that, when inserted/positioned into the gallbladder, have a sufficient portion to contact as much of the inner wall of a gallbladder so that a coil of thermal conductive wire about the inner mandrel can freeze the gallbladder without damaging surrounding anatomical structures or tissues (e.g., freezing or creating a hole in a duodenum). Furthermore, at least two of the pre-programmed loops may have varying sizes. In some cases, each pre-programmed loop varies in size to provide contact cause contact of a thermally conductive outer material to a sufficient portion of the inner wall of a gallbladder to freeze the gallbladder without damaging surrounding anatomical structures or tissues. The nitinol or para-aramid inner mandrel 700 also includes a longitudinal length that supports extension into the gallbladder for freezing the gallbladder.

Referring to FIG. 7B, a thermal conductive wire 702 in the form of a spiraling coil can be wrapped around the longitudinal length of the nitinol or para-aramid inner mandrel 700.

Referring to FIG. 7C, rings of thermal conductive wire 704 can be attached along the longitudinal length of the nitinol or para-aramid inner mandrel 700.

Referring to FIG. 7D-7F, a triangular shaped thermal conductive wire 706 can be attached to/positioned along a longitudinal length of the nitinol or para-aramid inner mandrel 700. Small gaps between the triangular shaped thermal conductive wire 706 provides good contact to the inner wall of a gallbladder while allowing for the deformation of the nitinol or para-aramid inner mandrel 700 during insertion into the gallbladder.

A double triangle profile silver ablation wire that can be used in the embodiment shown in FIGS. 7D-7F may be manufactured using two spools of oppositely oriented triangle profile wire that move down a longitudinal length of a nitinol or para-aramid inner mandrel 700 on a lathe chuck (that is oriented orthogonal to the triangle profile wire spools). The silver-wrapped nitinol or para-aramid inner mandrel 700 can be heated in a 500° C. furnace to shape (with the temperature setting to shape the nitinol or para-aramid in an air furnace being 600° C.). Note: the temperature is much less than the melting points of sterling silver, pure silver, and nitinol or para-aramid. Therefore, the triangle shape can occur before or after the silver is wrapped onto the core wire.

In some cases, the coil described with respect to FIGS. 7A-7F may be included in any of the embodiments of a cryoablation device described herein. In some cases, a compliant thermal conductive wire may be made of conductive material (e.g., silver) and have attributes (e.g., a diameter and/or hinges built into the longitudinal length) that give the compliant thermal conductive wire its “compliant” feature. In some cases, a sharp distal end is coupled to a distal end of the coil (e.g., to a distal end the nitinol or para-aramid inner mandrel 700 and/or a distal end of the thermal conductive wire.

As described herein, a nitinol or para-aramid inner mandrel 700 used as a guide to position a thermal conductive wire can be considered as a feature of the thermal conductive wire (e.g., because the nitinol or para-aramid inner mandrel 700 gives the thermal conductive wire its “compliant” feature). In some cases, the inner mandrel 700 is made of a high-strength synthetic fiber that is not nitinol or para-aramid yet is still compliant and biologically safe to use for insertion into a gallbladder. In some cases in which the inner mandrel 700 is made of para-aramid, the material of the para-aramid is more specifically poly-paraphenylene terephthalamide (e.g., Kevlar® by DuPont). In some cases, the thermal conductive wire is made of silver, copper, or some other highly thermally conductive material.

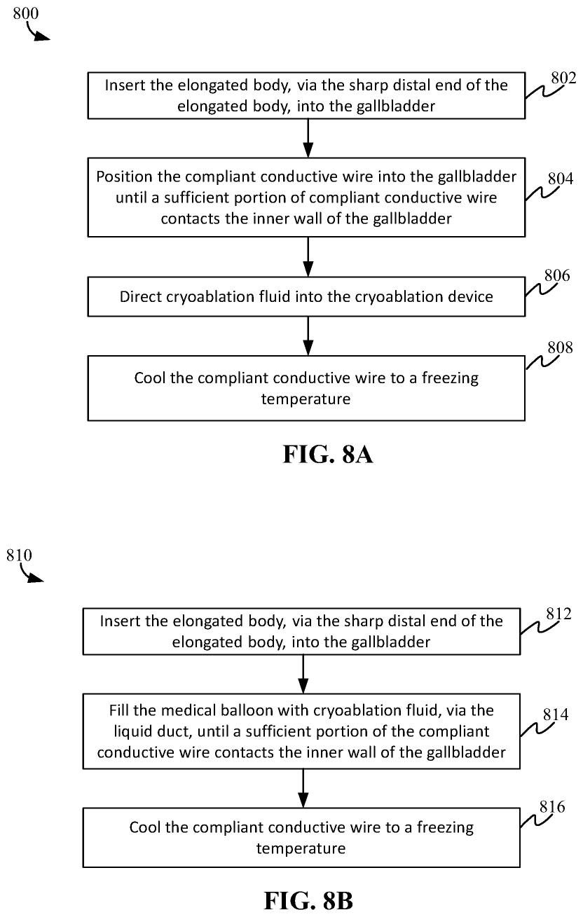

FIG. 8A illustrates a method of using a cryoablation device. Referring to FIG. 8A, a method 800 of using the cryoablation device (e.g., a cryoablation device without a medical balloon) includes inserting (802) the elongated body, via the sharp distal end of the elongated body, into the gallbladder; positioning (804) the compliant thermal conductive wire into the gallbladder until a sufficient portion of the compliant thermal conductive wire contacts the inner wall of the gallbladder; directing (806) cryoablation fluid into the cryoablation device; and cooling (808) the compliant thermal conductive wire to a freezing temperature, resulting in the gallbladder becoming frozen from the contact of the sufficient portion of the compliant thermal conductive wire to the inner wall of the gallbladder without damaging surrounding anatomical structures or tissues.

In some cases, positioning (804) the compliant thermal conductive wire into the gallbladder until a sufficient portion of the compliant thermal conductive wire contacts the inner wall of the gallbladder includes moving the compliant thermal conductive wire to a desired position that, when cooled (e.g., as described with respect to operation 808), allows a surgeon to direct formation of an ice ball that is formed during the freezing of the gallbladder and the proximal cystic duct. In other words, the position of the compliant thermal conductive wire in the gallbladder, during cooling (808), provides for formation of an ice ball to a shape and/or position around the compliant conductive material (which, in some cases as explained above, is in the shape of the gallbladder itself) as opposed to a shape and/or position of the most thermally conductive surrounding anatomical structure(s) (e.g., vessels). Furthermore, the amount of cryoablation fluid that is directed (806) into the cryoablation device to cool (808) the compliant thermal conductive wire to a freezing temperature may be aided by the use of an imaging device (e.g., a surgeon can use the imaging device to determine when the gallbladder is frozen) and may also depend on factors such as the size of the gallbladder, the diameter of the compliant thermal conductive wire, and/or the amount of contact the compliant thermal conductive wire makes with the inner wall of the gallbladder. In some cases, directing (806) the cryoablation fluid into the cryoablation device includes directing the cryoablation fluid, via the liquid duct, into a vaporization area of the elongated body and evacuating cryoablation gas, via an air duct, formed at the vaporization area.

FIG. 8B illustrates a method of using a cryoablation device with a medical balloon. Referring to FIG. 8B, a method (810) of using a cryoablation device with a medical balloon includes inserting (812) the elongated body, via the sharp distal end of the elongated body, into the gallbladder; filling (814) the medical balloon with cryoablation fluid, via the liquid duct, until a sufficient portion of the compliant thermal conductive wire contacts the inner wall of the gallbladder; and cooling (816) the compliant thermal conductive wire to a freezing temperature, resulting in the gallbladder becoming frozen from the contact of the sufficient portion of the compliant thermal conductive wire to the inner wall of the gallbladder without damaging surrounding anatomical structures or tissues.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A gallbladder cryoablation device, comprising:
an elongated body comprising a sharp distal end for insertion into a gallbladder, a liquid duct configured to direct a cryoablation fluid to a vaporization area in the elongated body, and an air duct configured to evacuate cryoablation gas formed at the vaporization area; and
a compliant thermal conductive wire associated with the elongated body and configured to extend from the elongated body and contact an inner wall of the gallbladder.

2. The gallbladder cryoablation device of claim 1, wherein the compliant thermal conductive wire is disposed between a proximal non-compliant band of thermal conductive material and a distal non-compliant band of thermal conductive material, wherein the distal non-compliant band of thermal conductive material is slidably attached to a distal portion of the elongated body and the proximal non-compliant band of thermal conductive material is affixed to a proximal portion of the elongated body.

3. The gallbladder cryoablation device of claim 2, wherein when the distal non-compliant band of thermal conductive material is slid from the distal portion of the elongated body towards the proximal portion of the elongated body, the compliant thermal conductive wire deforms outwardly towards the inner wall of the gallbladder.

4. The gallbladder cryoablation device of claim 1, wherein the compliant thermal conductive wire is disposed between a proximal non-compliant band of thermal conductive material and a distal non-compliant band of thermal conductive material, wherein the proximal non-compliant band of thermal conductive material is slidably attached to a proximal portion of the elongated body and the distal non-compliant band of thermal conductive material is affixed to a distal portion of the elongated body.

5. The gallbladder cryoablation device of claim 4, wherein when the proximal non-compliant band of thermal conductive material is slid from the proximal portion of the elongated body towards the distal portion of the elongated body, the compliant thermal conductive wire deforms outwardly towards the inner wall of the gallbladder.

6. The gallbladder cryoablation device of claim 1, further comprising a medical balloon positioned around the elongated body and in fluid communication with liquid duct of the elongated body.

7. The gallbladder cryoablation device of claim 6, wherein the compliant thermal conductive wire is positioned around the medical balloon.

8. The gallbladder cryoablation device of claim 7, wherein the compliant thermal conductive wire is configured to extend proportionally to match inflation of the medical balloon when cryoablation fluid is inserted, via the liquid duct of the elongated body, into the medical balloon.

9. The gallbladder cryoablation device of claim 8, wherein the medical balloon is in fluid communication with the air duct of the elongated body and configured to evacuate any cryoablation gas formed in the medical balloon.

10. The gallbladder cryoablation device of claim 1, wherein the compliant thermal conductive wire is a silver wire.

11. The gallbladder cryoablation device of claim 1, wherein the compliant thermal conductive wire comprises:
a compliant inner mandrel having a longitudinal length; and
a thermally conductive outer material coupled to the compliant inner mandrel along the longitudinal length of the compliant inner mandrel.

12. The gallbladder cryoablation device of claim 11, wherein the compliant inner mandrel comprises nitinol or para-aramid.

13. The gallbladder cryoablation device of claim 12, wherein the compliant inner mandrel comprises a plurality of pre-programmed loops configured to cause the thermally conductive outer material to contact a sufficient portion of the inner wall of the gallbladder to freeze the gallbladder.

14. The gallbladder cryoablation device of claim 13, wherein at least two of the plurality of loops have different diameters.

15. A method of using the cryoablation device of claim 1, comprising:
inserting the elongated body, via the sharp distal end of the elongated body, into the gallbladder;
positioning the compliant thermal conductive wire into the gallbladder until a sufficient portion of the compliant thermal conductive wire contacts the inner wall of the gallbladder;
directing the cryoablation fluid into the cryoablation device; and
cooling the compliant thermal conductive wire to a freezing temperature, resulting in the gallbladder becoming frozen from the contact of the sufficient portion of the compliant thermal conductive wire to the inner wall of the gallbladder without damaging surrounding anatomical structures or tissues.

16. A method of using the cryoablation device of claim 8, comprising:
inserting the elongated body, via the sharp distal end of the elongated body, into the gallbladder;
filling the medical balloon with cryoablation fluid, via the liquid duct, until a sufficient portion of the compliant thermal conductive wire contacts the inner wall of the gallbladder; and
cooling the compliant thermal conductive wire to a freezing temperature, resulting in the gallbladder becoming frozen from the contact of the sufficient portion of the compliant thermal conductive wire to the inner wall of the gallbladder without damaging surrounding anatomical structures or tissues.

17. A coil comprising:
a compliant inner mandrel having a longitudinal length; and
a thermally conductive outer material coupled to the compliant inner mandrel along the longitudinal length of the compliant inner mandrel.

18. The coil of claim 17, wherein the compliant inner mandrel is a high-strength synthetic fiber.

19. The coil of claim 18, wherein the high-strength synthetic fiber is nitinol or para-aramid.

20. The coil of claim 17, wherein the thermally conductive outer material is made of silver.

21. The coil of claim 17, wherein a cross-sectional diameter of the coil is between 1 millimeter and 4 millimeters.

22. The coil of claim 17, wherein the compliant inner mandrel comprises a plurality of pre-programmed loops configured to cause the thermally conductive outer material to contact a sufficient portion of an inner wall of a gallbladder to freeze the gallbladder.

23. The coil of claim 17, further comprising a sharp distal end coupled to a distal end of the compliant inner mandrel or a distal end of the thermally conductive outer material, wherein the sharp distal end is configured to pierce a gallbladder of a patient.
